(19) United States
(12) Patent Application Publication
(10) Pub. No.: US 2017/0000980 A1
(43) Pub. Date: Jan. 5, 2017

(54) ROBUST MINIATURE MAGNETIC SENSOR

(71) Applicant: Boston Scientific Scimed Inc., Maple Grove, MN (US)

(72) Inventor: John C. Potosky, San Jose, CA (US)

(21) Appl. No.: 15/197,667

(22) Filed: Jun. 29, 2016

Related U.S. Application Data
(60) Provisional application No. 62/186,367, filed on Jun. 30, 2015.

(51) Int. Cl. A61M 25/01; A61M 25/00; A61B 5/06

(52) U.S. Cl. CPC A61M 25/0127; A61B 5/062; A61M 25/0012

(57) ABSTRACT

A catheter assembly includes an elongated catheter shaft, a magnetic sensor, and one or more wires. The elongated catheter shaft has a shaft proximal end and a shaft distal end, and the magnetic sensor includes a coil having a coil proximal end and a coil distal end that is situated toward or at the shaft distal end. The one or more wires extend through the elongated catheter shaft and are situated adjacent the magnetic sensor. Each of the one or more wires is electrically coupled to the coil at the coil distal end.

### CROSS-REFERENCE TO RELATED APPLICATION

This application claims priority to Provisional Application No. 62/186,367, filed Jun. 30, 2015, which is herein incorporated by reference in its entirety.

### TECHNICAL FIELD

The present disclosure relates to magnetic sensors. More specifically, the disclosure relates to magnetic sensors in medical devices, such as catheters that include one or more magnetic sensors and methods of assembling the catheters.

### BACKGROUND

Catheters are medical devices that can be inserted into a patient's body to treat various medical conditions. Catheters can be tailored for use in cardiovascular, urological, gastrointestinal, neurovascular, and ophthalmic applications. Some catheters, referred to as ablation catheters, can be used to remove or terminate faulty electrical pathways in the heart.

Supraventricular tachycardia, ventricular tachycardia, and atrial fibrillation are conditions in the heart generally referred to as arrhythmias. During an arrhythmia, abnormal electrical signals, generated in the endocardial tissue, cause irregular beating of the heart. One method of treating these arrhythmias involves creating lesions within the chambers of the heart on the endocardium. These lesions are intended to stop the irregular beating of the heart by creating barriers between regions of the tissue. The barriers halt the passage through the heart of the abnormal currents generated in the endocardium. Energy, such as radio frequency (RF) energy, can be used to ablate tissue in the heart to form the lesion barriers and stop the flow of abnormal currents.

One apparatus for performing ablation is an ablation catheter having an ablative catheter tip. An electrode at the tip of an ablation catheter is placed inside the body and on the tissue to be ablated. A power supply generates electrical power, such as RF current, that is communicated between the electrode and a return electrode such that the energy ablates the tissue in the vicinity of the electrode.

Some catheters, including at least some ablation catheters, include sensors that permit tracking of the position and orientation of the catheter in the body of the patient. This can be used to more precisely position the catheter in the body of the patient. The necessarily small size of these sensors can make them mechanically fragile, which leads to failures and increased costs.

### SUMMARY

In an Example 1, a catheter assembly includes an elongated catheter shaft having a shaft proximal end and a shaft distal end. The catheter assembly is characterized by a magnetic sensor including a coil having a coil proximal end and a coil distal end situated toward or at the shaft distal end, and a pair of wires extending through the elongated catheter shaft, wherein at least one of the pair of wires is situated adjacent the coil from the coil proximal end to the coil distal end and coupled to the coil at the coil distal end.

In an Example 2, the catheter assembly according to Example 1 including a controller electrically coupled to the pair of wires toward or at the shaft proximal end. The controller is configured to determine the location of the shaft distal end in a patient based on signals from the magnetic sensor.

In an Example 3, the catheter assembly according to any of Examples 1 and 2, wherein the coil has a longitudinal axis from the coil proximal end to the coil distal end and one or more of the pair of wires has a straight portion that is parallel to the longitudinal axis of the coil from the coil proximal end to the coil distal end.

In an Example 4, the catheter assembly according to any of Examples 1-3, wherein the pair of wires are adjacent each other and the magnetic sensor.

In an Example 5, the catheter assembly according to any of Examples 1-3, wherein the pair of wires are adjacent the magnetic sensor and on opposing sides of the magnetic sensor.

In an Example 6, the catheter assembly according to any of Examples 1 and 2, wherein the at least one of the pair of wires is twisted around or spirals around the coil from the coil proximal end to the coil distal end.

In an Example 7, the catheter assembly according to any of Examples 1-6, wherein the pair of wires are twisted around each other proximal the magnetic sensor.

In an Example 8, the catheter assembly according to any of Examples 1-6, wherein the pair of wires are shielded straight wires proximal the magnetic sensor.

In an Example 9, the catheter assembly according to any of Examples 1-8, including a core having a high magnetic permeability, wherein the coil is wrapped around the core.

In an Example 10, the catheter assembly according to any of Examples 1-9, including at least one of encapsulation material that encapsulates the at least one of the pair of wires and the magnetic sensor, and an end cap that is distal the coil distal end and attached to the encapsulation material.

In an Example 11, a method of assembling a catheter assembly including an elongated catheter shaft having a shaft proximal end and a shaft distal end. The method being characterized by: providing a magnetic sensor including a coil having a coil proximal end and a coil distal end that is situated toward or at the shaft distal end; positioning at least one of a pair of wires adjacent the magnetic sensor from the coil proximal end to the coil distal end; electrically coupling the at least one of the pair of wires to the coil at the coil distal end; and encapsulating the magnetic sensor.

In an Example 12, the method according to Example 11, wherein positioning at least one of a pair of wires includes positioning a straight portion of the at least one of the pair of wires adjacent the magnetic sensor and a twisted portion of the pair of wires proximal the magnetic sensor.

In an Example 13, the method according to any of Examples 11 and 12, wherein positioning at least one of a pair of wires includes positioning each wire of the pair of wires adjacent the other wire and adjacent the magnetic sensor.

In an Example 14, the method according to any of Examples 11 and 12, wherein positioning at least one of a pair of wires includes positioning each wire of the pair of wires on opposing sides of the magnetic sensor and adjacent the magnetic sensor.

In an Example 15, the method according to Example 11, wherein positioning at least one of a pair of wires includes twisting the at least one of the pair of wires around the magnetic sensor.

In an Example 16, a catheter assembly including: an elongated catheter shaft having a shaft proximal end and a shaft distal end; a magnetic sensor including a coil having a coil proximal end and a coil distal end that is situated toward or at the shaft distal end; and one or more wires extending through the elongated catheter shaft and situated adjacent the magnetic sensor, the one or more wires electrically coupled to the coil at the coil distal end.

In an Example 17, the catheter assembly according to Example 16, including a controller electrically coupled to the one or more wires toward or at the shaft proximal end, the controller configured to determine the location of the shaft distal end in a patient based on signals from the magnetic sensor.

In an Example 18, the catheter assembly according to Example 16, wherein the coil has a longitudinal axis from the coil proximal end to the coil distal end and each of the one or more wires has a straight portion that is parallel to the longitudinal axis of the coil from the coil proximal end to the coil distal end.

In an Example 19, the catheter assembly according to Example 16, wherein the one or more wires are adjacent each other and the magnetic sensor.

In an Example 20, the catheter assembly according to Example 16, wherein the one or more wires are adjacent the magnetic sensor and on opposing sides of the magnetic sensor.

In an Example 21, the catheter assembly according to Example 16, wherein the one or more wires are twisted around or spiral around the coil from the coil proximal end to the coil distal end.

In an Example 22, the catheter assembly according to Example 16, wherein the one or more wires are twisted around each other proximal the magnetic sensor.

In an Example 23, the catheter assembly according to Example 16, wherein the one or more pair of wires are shielded straight twires that include a shield proximal the magnetic sensor.

In an Example 24, the catheter assembly according to Example 16, including a core having a high magnetic permeability, wherein the coil is wrapped around the core.

In an Example 25, the catheter assembly according to Example 16, including at least one of encapsulation material that encapsulates the one or more wires and the magnetic sensor in the encapsulation material, and an end cap that is distal the coil distal end and attached to the encapsulation material.

In an Example 26, a catheter assembly, including: an elongated catheter shaft having a shaft proximal end and a shaft distal end; a magnetic sensor including a coil having a longitudinal axis from a coil proximal end to a coil distal end that is situated at the shaft distal end; and a pair of wires extending through the elongated catheter shaft, from the shaft proximal end to the shaft distal end, wherein each of the pair of wires has a straight portion that is adjacent the coil and parallel to the longitudinal axis of the coil along the length of the magnetic sensor, the pair of wires electrically coupled to the coil at the coil distal end.

In an Example 27, the catheter assembly according to Example 26, wherein the pair of wires are adjacent each other and one side of the magnetic sensor.

In an Example 28, the catheter assembly according to Example 26, wherein the pair of wires are adjacent the magnetic sensor and on opposing sides of the magnetic sensor.

In an Example 29, the catheter assembly according to Example 26, wherein the pair of wires are twisted around each other proximal the magnetic sensor.

In an Example 30, the catheter assembly according to Example 26, including encapsulation material that encapsulates the pair of wires and the magnetic sensor in the encapsulation material.

In an Example 31, a method of assembling a catheter assembly including: providing an elongated catheter shaft having a shaft proximal end and a shaft distal end; providing a magnetic sensor including a coil having a coil proximal end and a coil distal end that is situated toward the shaft distal end; positioning at least one of a pair of wires adjacent the magnetic sensor from the coil proximal end to the coil distal end; and electrically coupling the at least one of the pair of wires to the coil at the coil distal end.

In an Example 32, the method according to Example 31, wherein positioning at least one of a pair of wires includes twisting the at least one of the pair of wires around the magnetic sensor.

In an Example 33, the method according to Example 31, wherein positioning at least one of a pair of wires comprises positioning a straight portion of each of the wires adjacent the magnetic sensor and a twisted portion of the pair of wires proximal the magnetic sensor.

In an Example 34, the method according to Example 31, wherein positioning at least one of a pair of wires comprises positioning each wire of the pair of wires adjacent the other wire and adjacent the magnetic sensor.

In an Example 35, the method according to Example 31, wherein positioning at least one of a pair of wires comprises positioning each wire of the pair of wires on opposing sides of the magnetic sensor and adjacent the magnetic sensor.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a diagram illustrating an ablation catheter assembly that includes a catheter tip and a magnetic sensor assembly, according to embodiments described in the disclosure.

FIG. 2 is a diagram illustrating a side view of a magnetic sensor assembly, according to embodiments described in the disclosure.

FIG. 3 is a diagram illustrating the magnetic sensor assembly of FIG. 2 taken along the line A-A in FIG. 2, according to embodiments described in the disclosure.

FIG. 4 is a diagram illustrating a side view of another magnetic sensor assembly, according to embodiments described in the disclosure.

FIG. 5 is a diagram illustrating the magnetic sensor assembly of FIG. 4 taken along the line B-B in FIG. 4, according to embodiments described in the disclosure.

FIG. 6 is a diagram illustrating another magnetic sensor assembly, according to embodiments described in the disclosure.

FIG. 7 is a diagram illustrating another magnetic sensor assembly, according to embodiments described in the disclosure.

FIG. 8 is a diagram illustrating another magnetic sensor assembly, according to embodiments described in the disclosure.

FIG. 9 is a flowchart diagram illustrating a method of assembling a catheter assembly, according to embodiments described in the disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the invention as defined by the appended claims.

### DETAILED DESCRIPTION

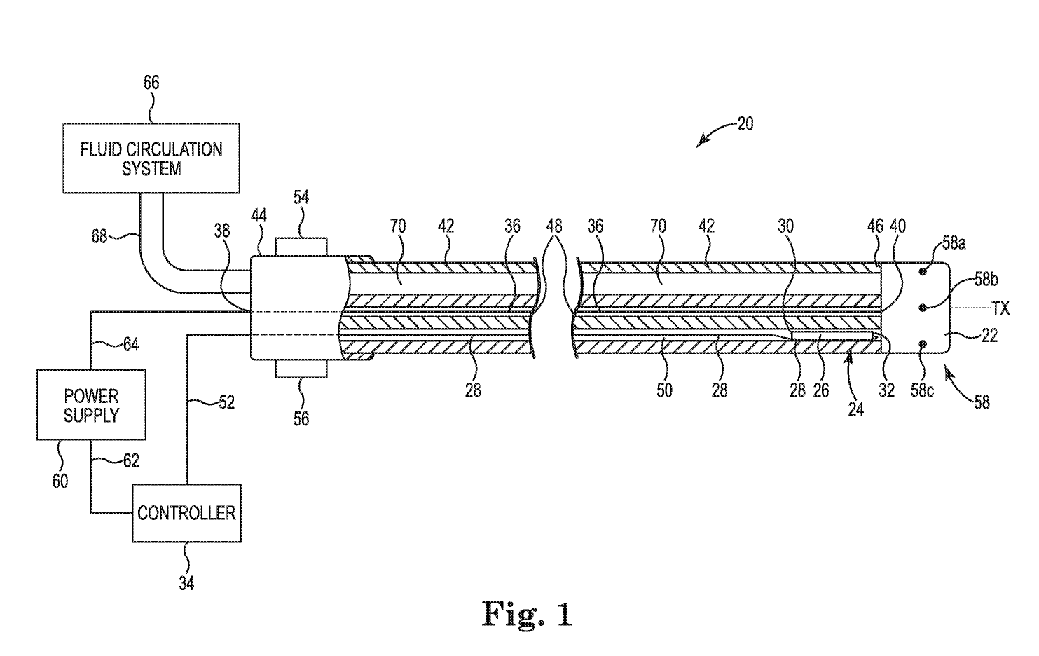

FIG. 1 is a diagram illustrating an ablation catheter assembly 20 that includes a catheter tip 22 and a magnetic sensor assembly 24, according to embodiments described in the disclosure. The magnetic sensor assembly 24 is situated near the catheter tip 22 for determining the location and orientation of the magnetic sensor assembly 24 and, by extension, the position of the catheter tip 22 in the body of a patient. In some embodiments, the magnetic sensor assembly 24 can be situated in the catheter tip 22.

The magnetic sensor assembly 24 includes a magnetic sensor 26 and one or more sensor lead wires 28. The magnetic sensor 26 has a sensor proximal end 30 and a sensor distal end 32. The magnetic sensor 26 may be electrically and mechanically coupled to the one or more sensor lead wires 28 at the sensor distal end 32. The one or more sensor lead wires 28 may be situated adjacent the magnetic sensor 26, from the sensor distal end 32 to the sensor proximal end 30, and the one or more sensor lead wires 28 may extend proximal the magnetic sensor 26. By coupling the one or more sensor lead wires 28 to the sensor distal end 32, instead of to the sensor proximal end 30, the connection between the magnetic sensor 26 and the one or more lead wires 28 may be made more robust, such that it may not be as easily pulled apart or broken by stresses on the magnetic sensor 26 and/or the one or more lead wires 28. The coupling may provide significant strain relief for the connections. In some embodiments, the magnetic sensor assembly 24 includes encapsulation material, such that the magnetic sensor 26 and the one or more sensor lead wires 28 are encapsulated in the encapsulation material at least from the sensor proximal end 30 to the sensor distal end 32. In some embodiments, the magnetic sensor 26 and the one or more sensor lead wires 28 are secured together, such as with an adhesive or plastic, at one or more locations in the magnetic sensor assembly 24.

The one or more sensor lead wires 28 may be electrically coupled to a controller 34 that receives signals from the magnetic sensor 26 and determines the position including the location and orientation of the magnetic sensor 26 and, by extension, the location and orientation of the catheter tip 22 based on the signals. In some embodiments, the controller 34 determines up to 5 degrees of freedom (DOF), including up/down, left/right, forward/backward, pitch, and yaw, based on the signals from the magnetic sensor 26. In other embodiments, the controller 34 determines up to 6 DOF, including up/down, left/right, forward/backward, pitch, yaw, and rotation of the catheter tip 22 around its longitudinal axis Tx, based on the signals from the magnetic sensor 26. In embodiments, the magnetic sensor 26 provides the signals in response to externally applied magnetic fields.

The ablation catheter assembly 20 includes a lead wire 36 having a proximal end 38 and a distal end 40. The lead wire 36 extends through an elongated catheter shaft 42 having a shaft proximal end 44 and a shaft distal end 46. The catheter tip 22 is attached to the shaft distal end 46 and the lead wire 36 extends through a wire lumen 48 in the elongated catheter shaft 42, from the shaft proximal end 44 to the catheter tip 22. An electrode (not shown) is attached to the distal end 40 of the lead wire 36 in the catheter tip 22. The catheter tip 22 is configured for treating tissue to be treated, where treatment of the tissue can include ablation of the tissue and/or creating lesions in the tissue. In embodiments, the catheter tip 22 bends or can be steered or bent to contact the tissue to be treated. In embodiments, the electrode and/or the catheter tip 22 includes stainless steel. In embodiments, the electrode and/or the catheter tip 22 includes platinum.

According to embodiments, the magnetic sensor assembly 24 is situated near the catheter tip 22 in a sensor lumen 50 in the elongated catheter shaft 42. The magnetic sensor 26 may be situated at or toward the shaft distal end 46 and the one or more sensor lead wires 28 extend through the sensor lumen 50 to the shaft proximal end 44. The controller 34 may be electrically coupled to the one or more sensor lead wires 28 at the shaft proximal end 44 via communications path 52.

In embodiments, the elongated catheter shaft 42 and the catheter tip 22 are each independently steerable or bendable. A shaft steering wire (not shown for clarity) may be connected to the shaft distal end 46 and to a shaft steering control 54 located at the shaft proximal end 44. Also, a catheter tip steering wire (not shown for clarity) may be connected to the catheter tip 22 and to a catheter tip steering control 56 at the shaft proximal end 44. Using the shaft steering control 54, the elongated catheter shaft 42 can be steered or bent to the tissue to be treated, and using the catheter tip steering control 56, the catheter tip 22 can be steered or bent to contact the tissue to be treated. The catheter tip 22 may be configured to be aligned to perform either spot ablation or linear ablation.

In embodiments, the ablation catheter assembly 20 includes mini electrodes 58 situated on at least one of the catheter tip 22 and the elongated catheter shaft 42. The mini-electrodes 58 can be used to indicate contact characteristics of the catheter tip 22 on the surface of the tissue. In some embodiments, three mini-electrodes 58a-58c are provided on the catheter tip 22.

The ablation catheter assembly 20 may include a power supply 60 that is communicatively coupled to the controller 34 via communications path 62 and electrically coupled to the proximal end 38 of the lead wire 36 via conductive pathway 64. The power supply 60 includes a source of energy that provides energy to the lead wire 36 and the electrode and the catheter tip 22. The controller 34 may be configured to control one or more characteristics of the energy delivered to the electrode and/or the catheter tip 22 to provide treatment to the tissue. In embodiments, the power supply 60 and the controller 34 provide high frequency energy, such as RF energy, to the electrode and the catheter tip 22 to ablate tissue and/or create lesions in the tissue.

The ablation catheter assembly 20 also may include a fluid circulation system 66 that is fluidically coupled to the elongated catheter shaft 42 at the shaft proximal end 44 via fluidic coupling 68. The elongated catheter shaft 42 may include one or more fluid conduits 70 that transfer fluid from the fluid circulation system 66 and pass the fluid through the elongated catheter shaft 42 to the catheter tip 22. The catheter tip 22 receives the fluid that cools the catheter tip 22. In embodiments, the fluid includes a saline solution.

In operation, the magnetic sensor assembly 24 and the catheter tip 22 are inserted into the body of a patient and the catheter tip 22 is positioned against or next to the tissue to be treated. The magnetic sensor 26 provides signals to the controller 34 that determines the position and/or orientation of the catheter tip 22 in the body based on the signals. This position information may be used to position the catheter tip 22 against or next to the tissue to be treated. The fluid circulation system 66 provides the fluid through the elongated catheter shaft 42 to the catheter tip 22, and the power supply 60 and the controller 34 provide electrical energy to the electrode and the catheter tip 22 to treat the tissue. Treatment of the tissue can include spot or linear ablation of the tissue and/or creating lesions in the tissue.

By coupling the one or more sensor lead wires 28 to the sensor distal end 32, instead of to the sensor proximal end 30, the connection between the magnetic sensor 26 and the one or more lead wires 28 may be made more robust, such that it is not easily pulled apart during use, such as while positioning the catheter tip 22 against or next to the tissue to be treated, or during assembly of the ablation catheter assembly 20. In other embodiments, the magnetic sensor assembly 24 can be used in other devices, including other medical devices and/or other catheters.

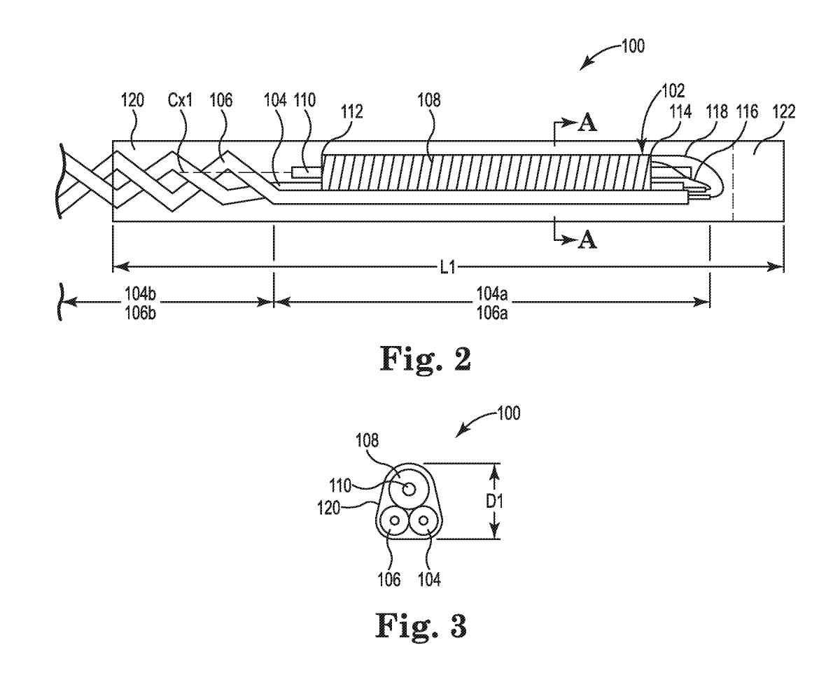

FIGS. 2 and 3 are diagrams illustrating a magnetic sensor assembly 100, according to embodiments described in the disclosure. FIG. 2 is a diagram illustrating a side view of the magnetic sensor assembly 100, according to embodiments described in the disclosure. FIG. 3 is a diagram illustrating the magnetic sensor assembly 100 taken along the line A-A in FIG. 2, according to embodiments described in the disclosure. The magnetic sensor assembly 100 can be used in the ablation catheter assembly 20 of FIG. 1. Also, the magnetic sensor assembly 100 may be, include, or be similar to the magnetic sensor assembly 24. In some embodiments, the magnetic sensor assembly 100 has a length L1 of 1 centimeter. In some embodiments, the magnetic sensor assembly 100 has a closest circular fit diameter D1 of 0.5 millimeters.

According to embodiments, the magnetic sensor assembly 100 includes a magnetic sensor 102 and two sensor lead wires 104 and 106 electrically coupled to the magnetic sensor 102. The magnetic sensor 102 includes a magnetic sensor coil 108 and a magnetic core 110. The sensor coil 108 may include a number of turns of wire and has a coil proximal end 112 and a coil distal end 114. The wire of the sensor coil 108 may be wound or wrapped around the magnetic core 110. In some embodiments, the sensor coil 108 includes wire in a range of from 52 to 58 gauge wire. In some embodiments, the sensor coil 108 includes a number of turns of wire in a range of from 1500 to 3500 turns of wire. In embodiments, the magnetic core 110 is made from a high magnetic permeability material, such as HyMu80, which greatly increases sensitivity of the magnetic sensor assembly 100. In embodiments, the two sensor lead wires 104 and 106 include wire in a range of from 36 to 40 gauge wire.

In illustrated embodiments, the sensor coil 108 may provide two coil lead wires 116 and 118 at the coil distal end 114. The two coil lead wires 116 and 118 electrically and mechanically couple the sensor coil 108 to the two sensor lead wires 104 and 106. The coil lead wire 116 is electrically and mechanically coupled to sensor lead wire 104, and the coil lead wire 118 is electrically and mechanically coupled to sensor lead wire 106. In embodiments, at least one of the two coil lead wires 116 and 118 is coupled to the corresponding sensor lead wire 104 and 106 by soldering. In embodiments, at least one of the two coil lead wires 116 and 118 is coupled to the corresponding sensor lead wire 104 and 106 by an adhesive. In embodiments, at least one of the two coil lead wires 116 and 118 is coupled to the corresponding sensor lead wire 104 and 106 by laser wire attachment.

The two sensor lead wires 104 and 106 may be situated adjacent the magnetic sensor coil 108, from the coil distal end 114 to the coil proximal end 112, and the two sensor lead wires 104 and 106 extend proximal the sensor coil 108. Also, the two sensor lead wires 104 and 106 may be situated adjacent each other on one side of the magnetic sensor coil 108. Each of the two sensor lead wires 104 and 106 may include a straight portion 104a and 106a that is situated adjacent the magnetic sensor coil 108, at least from the coil distal end 114 to the coil proximal end 112. Also, the sensor coil 108 may have a longitudinal axis Cx1 and each of the straight portions 104a and 106a may be parallel to the longitudinal axis Cx1 of the sensor coil 108 from the coil proximal end 112 to the coil distal end 114. In addition, each of the two sensor lead wires 104 and 106 may include a twisted pair portion 104b and 106b in the pair of sensor lead wires 104 and 106 that is situated proximal to the magnetic sensor coil 108. The twisted pair portions 104b and 106b reduce sensor signal noise. In other embodiments, the sensor lead wires 104 and 106 do not include the twisted pair portions 104b and 106b, instead, the sensor lead wires 104 and 106 are straight lead wires proximal the magnetic sensor coil 108 and shielded with a conductor, such as a braid of copper, which is advantageous for improving lead tensile strength and reducing sensor signal noise. This shielding can extend to the proximal end 112 or all the way to the distal end 114 of the magnetic sensor coil 108 and it can cover the connections between the coil lead wires 116 and 118 and the sensor lead wires 104 and 106. In some embodiments, the shield is electrically isolated from the sensor lead wires 104 and 106, the magnetic sensor coil 108, and/or the magnetic core 110.

In embodiments, the magnetic sensor assembly 100 may include an encapsulation material 120 that is wrapped around the magnetic sensor 102 and the two sensor lead wires 104 and 106, at least from the coil proximal end 112 to the coil distal end 114. Optionally, an end cap 122 that is distal the coil distal end 114, may be attached to the encapsulation material 120 to seal the encapsulation material 120 at the coil distal end 114. In embodiments, the magnetic sensor 102 and the two sensor lead wires 104 and 106 are slid into a tube of encapsulation material 120. In embodiments, the encapsulation material 120 is a shrink wrap material. In embodiments, the sensor coil 108 and the two sensor lead wires 104 and 106 are secured together, such as with an adhesive or plastic, at one or more locations along the sensor coil 108. In some embodiments, the end cap 122 includes at least one of epoxy, polyimide, and room temperature vulcanization silicone.

In assembling a catheter assembly such as ablation catheter assembly 24 of FIG. 1, the magnetic sensor assembly 100 may be fit into the sensor lumen 50 and secured in place, such as with an adhesive. The coil distal end 114 may be situated at the shaft distal end 46 and the two sensor lead wires 104 and 106 extend through the sensor lumen 50 to the shaft proximal end 44. The two sensor lead wires 104 and 106 may be electrically coupled to the controller 34 at the shaft proximal end 44. In embodiments, the controller 34 receives the signals from the magnetic sensor 102 for determining the position of the catheter tip 22, where the magnetic sensor 102 provides the signals in response to externally applied magnetic fields.

Using a single coil magnetic sensor, such as magnetic sensor 102, the controller 34 can determine up to 5 DOF, including up/down, left/right, forward/backward, pitch, and yaw, based on the signals from the magnetic sensor 102. In other embodiments, such as where the magnetic sensor includes two or more magnetic sensor coils that are not parallel with each other along their longitudinal axes, the controller 34 can determine up to 6 DOF, including up/down, left/right, forward/backward, pitch, yaw, and rotation of the catheter tip 22 around its longitudinal axis Tx, based on the signals from the magnetic sensor.

Coupling the two sensor lead wires 104 and 106 to the two coil lead wires 116 and 118 at the coil distal end 114 may provide significant strain relief for the connections. By coupling the pair of sensor lead wires 104 and 106 to the two coil lead wires 116 and 118 at the coil distal end 114, instead of to the coil proximal end 112, the connections between the magnetic sensor 102 and the two sensor lead wires 104 and 106 may be made more robust, such that they are not easily pulled apart or broken by stresses on the magnetic sensor 102 and the two sensor lead wires 104 and 106. The encapsulation material 120 may increase the durability of the connections between the magnetic sensor 102 and the two sensor lead wires 104 and 106. These features may reduce risk to the patient and improve product quality and profitability.

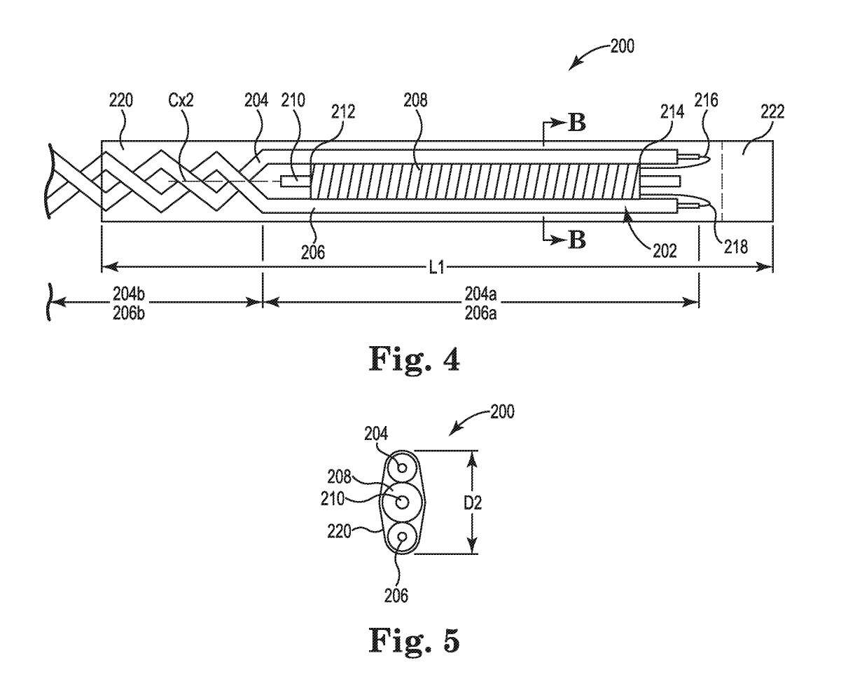

FIGS. 4 and 5 are diagrams illustrating another magnetic sensor assembly 200, according to embodiments described in the disclosure. FIG. 4 is a diagram illustrating a side view of the magnetic sensor assembly 200, according to embodiments described in the disclosure. FIG. 5 is a diagram illustrating the magnetic sensor assembly 200 taken along the line B-B in FIG. 4, according to embodiments described in the disclosure. The magnetic sensor assembly 200 is similar to the magnetic sensor assembly 100 of FIGS. 2 and 3, with the exception that the two sensor lead wires 204 and 206 are situated on opposing sides of the magnetic sensor coil 208, and not adjacent each other.

The magnetic sensor assembly 200 can be used in the ablation catheter assembly 20 of FIG. 1. Also, the magnetic sensor assembly 200 may be, include, or be similar to the magnetic sensor assembly 24. In some embodiments, the magnetic sensor assembly 200 has a length L2 of 1 centimeter. In some embodiments, the magnetic sensor assembly 200 has a closest circular fit diameter D2 of 0.5 millimeters.

The magnetic sensor assembly 200 includes a magnetic sensor 202 and two sensor lead wires 204 and 206 electrically coupled to the magnetic sensor 202. The magnetic sensor 202 includes a magnetic sensor coil 208 and a magnetic core 210. The sensor coil 208 includes a number of turns of wire and has a coil proximal end 212 and a coil distal end 214. The wire of the sensor coil 208 is wound or wrapped around the magnetic core 210. In some embodiments, the sensor coil 208 includes wire in a range of from 52 to 58 gauge wire. In some embodiments, the sensor coil 208 includes a number of turns of wire in a range of from 1500 to 3500 turns of wire. In embodiments, the magnetic core 210 is made from a high magnetic permeability material, such as HyMu80, which greatly increases sensitivity of the magnetic sensor assembly 200. In embodiments, the two sensor lead wires 204 and 206 include wire in a range of from 36 to 40 gauge wire.

The sensor coil 208 provides two coil lead wires 216 and 218 at the coil distal end 214. The two coil lead wires 216 and 218 electrically and mechanically couple the sensor coil 208 to the two sensor lead wires 204 and 206. The coil lead wire 216 is electrically and mechanically coupled to sensor lead wire 204, and the coil lead wire 218 is electrically and mechanically coupled to sensor lead wire 206. In some embodiments, at least one of the two coil lead wires 216 and 218 is coupled to the corresponding sensor lead wire 204 and 206 by soldering. In some embodiments, at least one of the two coil lead wires 216 and 218 is coupled to the corresponding sensor lead wire 204 and 206 by an adhesive. In some embodiments, at least one of the two coil lead wires 216 and 218 is coupled to the corresponding sensor lead wire 204 and 206 by laser wire attachment.

The two sensor lead wires 204 and 206 are situated adjacent the magnetic sensor coil 208, from the coil distal end 214 to the coil proximal end 212, and the two sensor lead wires 204 and 206 extend proximal the sensor coil 208. Also, the two sensor lead wires 204 and 206 are situated on opposing sides of the magnetic sensor coil 208 and not adjacent each other on one side of the magnetic sensor coil 208. Each of the two sensor lead wires 204 and 206 includes a straight portion 204a and 206a that is situated adjacent the magnetic sensor coil 208, at least from the coil distal end 214 to the coil proximal end 212. Also, the sensor coil 208 has a longitudinal axis Cx2 and each of the straight portions 204a and 206a is parallel to the longitudinal axis Cx2 of the sensor coil 208 from the coil proximal end 212 to the coil distal end 214. In addition, each of the two sensor lead wires 204 and 206 includes a twisted pair portion 204b and 206b in the pair of sensor lead wires 204 and 206 that is situated proximal to the magnetic sensor coil 208. The twisted pair portions 204b and 206b reduce sensor signal noise. In other embodiments, the sensor lead wires 204 and 206 do not include the twisted pair portions 204b and 206b, instead, the sensor lead wires 204 and 206 are straight lead wires proximal the magnetic sensor coil 208 and shielded with a conductor, such as a braid of copper, which is advantageous for improving lead tensile strength and reducing sensor signal noise. This shielding can extend to the proximal end 212 or all the way to the distal end 214 of the magnetic sensor coil 208 and it can cover the connections between the coil lead wires 216 and 218 and the sensor lead wires 204 and 206. In some embodiments, the shield is electrically isolated from the sensor lead wires 204 and 206, the magnetic sensor coil 208, and/or the magnetic core 210.

The magnetic sensor assembly 200 includes an encapsulation material 220 that is wrapped around the magnetic sensor 202 and the two sensor lead wires 204 and 206, at least from the coil proximal end 212 to the coil distal end 214. Optionally, an end cap 222 that is distal the coil distal end 214, is attached to the encapsulation material 220 to seal the encapsulation material 220 at the coil distal end 214. In some embodiments, the magnetic sensor 202 and the two sensor lead wires 204 and 206 are slid into a tube of encapsulation material 220. In some embodiments, the encapsulation material 220 is a shrink wrap material. In some embodiments, the sensor coil 208 and the two sensor lead wires 204 and 206 are secured together, such as with an adhesive or plastic, at one or more locations along the sensor coil 208. In some embodiments, the end cap 222 includes at least one of epoxy, polyimide, and room temperature vulcanization silicone.

In assembling a catheter assembly such as ablation catheter assembly 24 of FIG. 1, the magnetic sensor assembly 200 is fit into the sensor lumen 50 and secured in place, such as with an adhesive. The coil distal end 214 is situated at the shaft distal end 46 and the two sensor lead wires 204 and 206 extend through the sensor lumen 50 to the shaft proximal end 44. The two sensor lead wires 204 and 206 are electrically coupled to the controller 34 at the shaft proximal end 44. The controller 34 receives the signals from the magnetic sensor 202 for determining the position of the catheter tip 22, where the magnetic sensor 202 provides the signals in response to externally applied magnetic fields.

Using a single coil magnetic sensor, such as magnetic sensor 202, the controller 34 can determine up to 5 DOF, including up/down, left/right, forward/backward, pitch, and yaw, based on the signals from the magnetic sensor 202. In other embodiments, where the magnetic sensor includes two or more magnetic sensor coils that are not parallel with each other along their longitudinal axes, the controller 34 can determine up to 6 DOF, including up/down, left/right, forward/backward, pitch, yaw, and rotation of the catheter tip 22 around its longitudinal axis Tx, based on the signals from the magnetic sensor.

Coupling the two sensor lead wires 204 and 206 to the two coil lead wires 216 and 218 at the coil distal end 214 may provide significant strain relief for the connections. By coupling the pair of sensor lead wires 204 and 206 to the two coil lead wires 216 and 218 at the coil distal end 214, instead of to the coil proximal end 212, the connections between the magnetic sensor 202 and the two sensor lead wires 204 and 206 may be made more robust, such that they are not easily pulled apart or broken by stresses on the magnetic sensor 202 and the two sensor lead wires 204 and 206. The encapsulation material 220 may increase the durability of the connections between the magnetic sensor 202 and the two sensor lead wires 204 and 206. These features reduce risk to the patient and improve product quality and profitability.

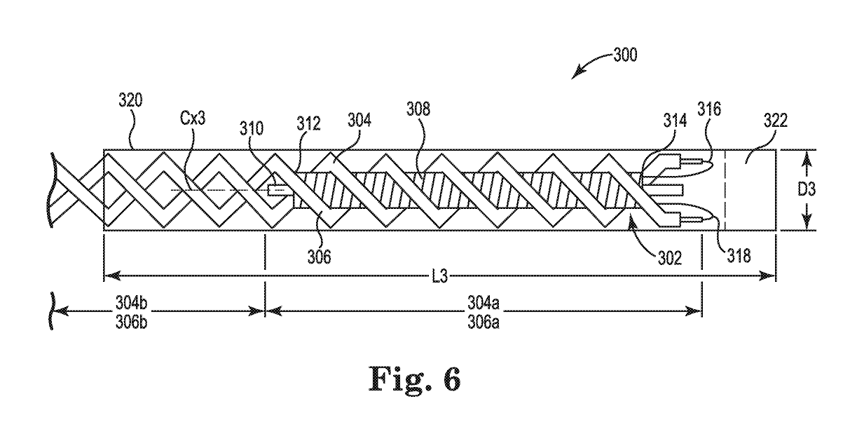

FIG. 6 is a diagram illustrating another magnetic sensor assembly 300, according to embodiments described in the disclosure. The magnetic sensor assembly 300 is similar to the magnetic sensor assemblies 100 and 200, with the exception that the two sensor lead wires 304 and 306 are twisted around or spiral around the magnetic sensor coil 308.

The magnetic sensor assembly 300 can be used in the ablation catheter assembly 20 of FIG. 1. Also, the magnetic sensor assembly 300 may be, include, or be similar to the magnetic sensor assembly 24. In some embodiments, the magnetic sensor assembly 300 has a length L3 of 1 centimeter. In some embodiments, the magnetic sensor assembly 300 has a closest circular fit diameter D3 of 0.5 millimeters.

The magnetic sensor assembly 300 includes a magnetic sensor 302 and two sensor lead wires 304 and 306 electrically coupled to the magnetic sensor 302. The magnetic sensor 302 includes a magnetic sensor coil 308 and a magnetic core 310. The sensor coil 308 includes a number of turns of wire and has a coil proximal end 312 and a coil distal end 314. The wire of the sensor coil 308 is wound or wrapped around the magnetic core 310. In some embodiments, the sensor coil 308 includes wire in a range of from 52 to 58 gauge wire. In some embodiments, the sensor coil 308 includes a number of turns of wire in a range of from 1500 to 3500 turns of wire. In embodiments, the magnetic core 310 is made from a high magnetic permeability material, such as HyMu80, which greatly increases sensitivity of the magnetic sensor assembly 300. In embodiments, the two sensor lead wires 304 and 306 include wire in a range of from 36 to 40 gauge wire.

The sensor coil 308 provides two coil lead wires 316 and 318 at the coil distal end 314. The two coil lead wires 316 and 318 electrically and mechanically couple the sensor coil 308 to the two sensor lead wires 304 and 306. The coil lead wire 316 is electrically and mechanically coupled to sensor lead wire 304, and the coil lead wire 318 is electrically and mechanically coupled to sensor lead wire 306. In some embodiments, at least one of the two coil lead wires 316 and 318 is coupled to the corresponding sensor lead wire 304 and 306 by soldering. In some embodiments, at least one of the two coil lead wires 316 and 318 is coupled to the corresponding sensor lead wire 304 and 306 by an adhesive. In some embodiments, at least one of the two coil lead wires 316 and 318 is coupled to the corresponding sensor lead wire 304 and 306 by laser wire attachment.

The two sensor lead wires 304 and 306 are wrapped or twisted around the magnetic sensor coil 308, from the coil distal end 314 to the coil proximal end 312, at lead wire portions 304a and 306a. The sensor coil 308 has a longitudinal axis Cx3 and the two sensor lead wires 304 and 306 extend proximal the sensor coil 308 at twisted pair portions 304b and 306b. The twisted pair portions 304b and 306b reduce sensor signal noise. In other embodiments, the sensor lead wires 304 and 306 do not include the twisted pair portions 304b and 306b, instead, the sensor lead wires 304 and 306 are straight lead wires proximal the magnetic sensor coil 308 and shielded with a conductor, such as a braid of copper, which is advantageous for improving lead tensile strength and reducing sensor signal noise. This shielding can extend to the proximal end 312 or all the way to the distal end 314 of the magnetic sensor coil 308 and it can cover the connections between the coil lead wires 316 and 318 and the sensor lead wires 304 and 306. In some embodiments, the shield is electrically isolated from the sensor lead wires 304 and 306, the magnetic sensor coil 308, and/or the magnetic core 310.

The magnetic sensor assembly 300 includes an encapsulation material 320 that is wrapped around the magnetic sensor 302 and the two sensor lead wires 304 and 306, at least from the coil proximal end 312 to the coil distal end 314. Optionally, an end cap 322 that is distal the coil distal end 314, is attached to the encapsulation material 320 to seal the encapsulation material 320 at the coil distal end 314. In some embodiments, the magnetic sensor 302 and the two sensor lead wires 304 and 306 are slid into a tube of encapsulation material 320. In some embodiments, the encapsulation material 320 is a shrink wrap material. In some embodiments, the sensor coil 308 and the two sensor lead wires 304 and 306 are secured together, such as with an adhesive or plastic, at one or more locations along the sensor coil 308. In some embodiments, the end cap 322 includes at least one of epoxy, polyimide, and room temperature vulcanization silicone.

In assembling a catheter assembly such as ablation catheter assembly 24 of FIG. 1, the magnetic sensor assembly 300 is fit into the sensor lumen 50 and secured in place, such as with an adhesive. The coil distal end 314 is situated at the shaft distal end 46 and the two sensor lead wires 304 and 306 extend through the sensor lumen 50 to the shaft proximal end 44. The two sensor lead wires 304 and 306 are electrically coupled to the controller 34 at the shaft proximal end 44. The controller 34 receives the signals from the magnetic sensor 302 for determining the position of the catheter tip 22, where the magnetic sensor 302 provides the signals in response to externally applied magnetic fields.

Using a single coil magnetic sensor, such as magnetic sensor 302, the controller 34 can determine up to 5 DOF, including up/down, left/right, forward/backward, pitch, and yaw, based on the signals from the magnetic sensor 302. In other embodiments, where the magnetic sensor includes two or more magnetic sensor coils that are not parallel with each other along their longitudinal axes, the controller 34 can determine up to 6 DOF, including up/down, left/right, forward/backward, pitch, yaw, and rotation of the catheter tip 22 around its longitudinal axis Tx, based on the signals from the magnetic sensor.

Coupling the two sensor lead wires 304 and 306 to the two coil lead wires 316 and 318 at the coil distal end 314 may provide significant strain relief for the connections. By coupling the pair of sensor lead wires 304 and 306 to the two coil lead wires 316 and 318 at the coil distal end 314, instead of to the coil proximal end 312, the connections between the magnetic sensor 302 and the two sensor lead wires 304 and 306 may be made more robust, such that they are not easily pulled apart or broken by stresses on the magnetic sensor 302 and the two sensor lead wires 304 and 306. The encapsulation material 320 may increase the durability of the connections between the magnetic sensor 302 and the two sensor lead wires 304 and 306. These features reduce risk to the patient and improve product quality and profitability.

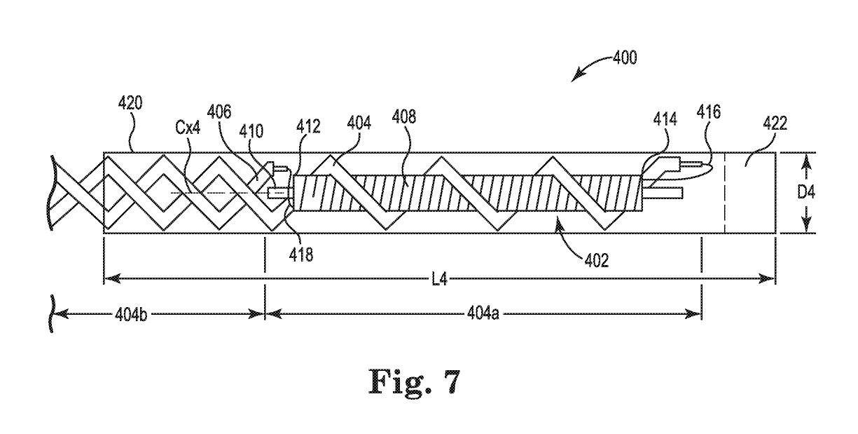

FIG. 7 is a diagram illustrating another magnetic sensor assembly 400, according to embodiments described in the disclosure. The magnetic sensor assembly 400 is similar to the magnetic sensor assemblies 100, 200, and 300, with the exception that sensor lead wire 404 is twisted around or spirals around the magnetic sensor coil 408 and sensor lead wire 406 is attached to the magnetic sensor coil 408 at the proximal end of the magnetic sensor coil 408.

The magnetic sensor assembly 400 can be used in the ablation catheter assembly 20 of FIG. 1. Also, the magnetic sensor assembly 400 may be, include, or be similar to the magnetic sensor assembly 24. In some embodiments, the magnetic sensor assembly 400 has a length L4 of 1 centimeter. In some embodiments, the magnetic sensor assembly 400 has a closest circular fit diameter D4 of 0.5 millimeters.

The magnetic sensor assembly 400 includes a magnetic sensor 402 and two sensor lead wires 404 and 406 electrically coupled to the magnetic sensor 402. The magnetic sensor 402 includes a magnetic sensor coil 408 and a magnetic core 410. The sensor coil 408 includes a number of turns of wire and has a coil proximal end 412 and a coil distal end 414. The wire of the sensor coil 408 is wound or wrapped around the magnetic core 410. In some embodiments, the sensor coil 408 includes wire in a range of from 52 to 58 gauge wire. In some embodiments, the sensor coil 408 includes a number of turns of wire in a range of from 1500 to 3500 turns of wire. In embodiments, the magnetic core 410 is made from a high magnetic permeability material, such as HyMu80, which greatly increases sensitivity of the magnetic sensor assembly 400. In embodiments, the two sensor lead wires 404 and 406 include wire in a range of from 36 to 40 gauge wire.

The sensor coil 408 provides coil lead wire 416 at the coil distal end 414 and coil lead wire 418 at the coil proximal end 412. The two coil lead wires 416 and 418 electrically and mechanically couple the sensor coil 408 to the two sensor lead wires 404 and 406. The coil lead wire 416 is electrically and mechanically coupled to sensor lead wire 404, and the coil lead wire 418 is electrically and mechanically coupled to sensor lead wire 406. In some embodiments, at least one of the two coil lead wires 416 and 418 is coupled to the corresponding sensor lead wire 404 and 406 by soldering. In some embodiments, at least one of the two coil lead wires 416 and 418 is coupled to the corresponding sensor lead wire 404 and 406 by an adhesive. In some embodiments, at least one of the two coil lead wires 416 and 418 is coupled to the corresponding sensor lead wire 404 and 406 by laser wire attachment.

The sensor lead wire 404 is wrapped or twisted around the magnetic sensor coil 408, from the coil distal end 414 to the coil proximal end 412, at lead wire portion 404a. The sensor coil 408 has a longitudinal axis Cx4 and the two sensor lead wires 404 and 406 extend proximal the sensor coil 408 in a twisted pair at twisted pair portion 404b. The twisted pair at twisted pair portion 404b reduces sensor signal noise. In other embodiments, the sensor lead wires 404 and 406 do not include the twisted pair portion at 404b, instead, the sensor lead wires 404 and 406 are straight lead wires proximal the magnetic sensor coil 408 and shielded with a conductor, such as a braid of copper, which is advantageous for improving lead tensile strength and reducing sensor signal noise. This shielding can extend to the proximal end 412 or all the way to the distal end 414 of the magnetic sensor coil 408 and it can cover the connections between the coil lead wires 416 and 418 and the sensor lead wires 404 and 406. In some embodiments, the shield is electrically isolated from the sensor lead wires 404 and 406, the magnetic sensor coil 408, and/or the magnetic core 410.

The magnetic sensor assembly 400 includes an encapsulation material 420 that is wrapped around the magnetic sensor 402 and, in at least some embodiments, around portions of the two sensor lead wires 404 and 406. Optionally, an end cap 422 that is distal the coil distal end 414, is attached to the encapsulation material 420 to seal the encapsulation material 420 at the coil distal end 414. In some embodiments, the magnetic sensor 402 and the two sensor lead wires 404 and 406 are slid into a tube of encapsulation material 420. In some embodiments, the encapsulation material 420 is a shrink wrap material. In some embodiments, the sensor coil 408 and the sensor lead wire 404 are secured together, such as with an adhesive or plastic, at one or more locations along the sensor coil 408. In some embodiments, the end cap 422 includes at least one of epoxy, polyimide, and room temperature vulcanization silicone.

In assembling a catheter assembly such as ablation catheter assembly 24 of FIG. 1, the magnetic sensor assembly 400 is fit into the sensor lumen 50 and secured in place, such as with an adhesive. The coil distal end 414 is situated at the shaft distal end 46 and the two sensor lead wires 404 and 406 extend through the sensor lumen 50 to the shaft proximal end 44. The two sensor lead wires 404 and 406 are electrically coupled to the controller 34 at the shaft proximal end 44. The controller 34 receives the signals from the magnetic sensor 402 for determining the position of the catheter tip 22, where the magnetic sensor 402 provides the signals in response to externally applied magnetic fields.

Using a single coil magnetic sensor, such as magnetic sensor 402, the controller 34 can determine up to 5 DOF, including up/down, left/right, forward/backward, pitch, and yaw, based on the signals from the magnetic sensor 402. In other embodiments, where the magnetic sensor includes two or more magnetic sensor coils that are not parallel with each other along their longitudinal axes, the controller 34 can determine up to 6 DOF, including up/down, left/right, forward/backward, pitch, yaw, and rotation of the catheter tip 22 around its longitudinal axis Tx, based on the signals from the magnetic sensor.

Coupling the sensor lead wire 404 to the coil lead wire 416 at the coil distal end 414 may provide significant strain relief for the connections. By coupling the sensor lead wire 404 to the coil lead wire 416 at the coil distal end 414, instead of to the coil proximal end 412, the connection between the magnetic sensor 402 and the sensor lead wire 404 may be made more robust, such that it is not easily pulled apart or broken by stresses on the magnetic sensor 402 and the sensor lead wire 404. The encapsulation material 420 may increase the durability of the connections between the magnetic sensor 402 and the two sensor lead wires 404 and 406. These features reduce risk to the patient and improve product quality and profitability.

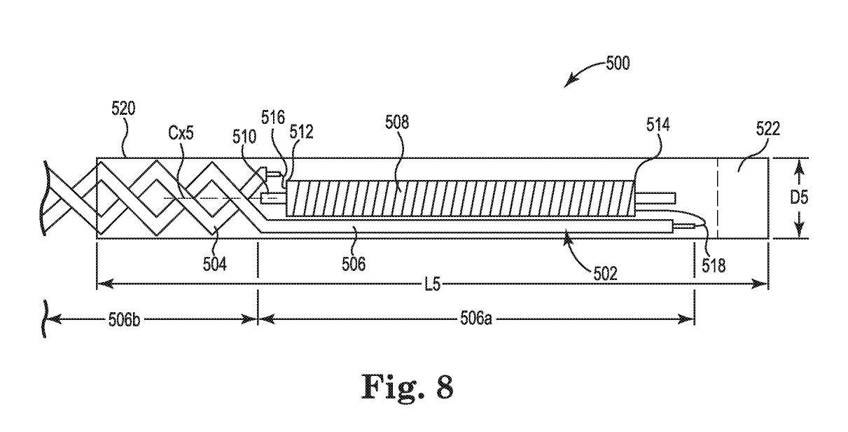

FIG. 8 is a diagram illustrating another magnetic sensor assembly 500, according to embodiments described in the disclosure. The magnetic sensor assembly 500 is similar to the magnetic sensor assemblies 100, 200, 300, and 400, with the exception that sensor lead wire 506 is straight and parallel to the magnetic sensor coil 508 along the length of the magnetic sensor coil 508 and sensor lead wire 504 is attached to the magnetic sensor coil 508 at the proximal end of the magnetic sensor coil 508.

The magnetic sensor assembly 500 can be used in the ablation catheter assembly 20 of FIG. 1. Also, the magnetic sensor assembly 500 may be, include, or be similar to the magnetic sensor assembly 24. In some embodiments, the magnetic sensor assembly 500 has a length L5 of 1 centimeter. In some embodiments, the magnetic sensor assembly 500 has a closest circular fit diameter D5 of 0.5 millimeters.

The magnetic sensor assembly 500 includes a magnetic sensor 502 and two sensor lead wires 504 and 506 electrically coupled to the magnetic sensor 502. The magnetic sensor 502 includes a magnetic sensor coil 508 and a magnetic core 510. The sensor coil 508 includes a number of turns of wire and has a coil proximal end 512 and a coil distal end 514. The wire of the sensor coil 508 is wound or wrapped around the magnetic core 510. In some embodiments, the sensor coil 508 includes wire in a range of from 52 to 58 gauge wire. In some embodiments, the sensor coil 508 includes a number of turns of wire in a range of from 1500 to 3500 turns of wire. In embodiments, the magnetic core 510 is made from a high magnetic permeability material, such as HyMu80, which greatly increases sensitivity of the magnetic sensor assembly 500. In embodiments, the two sensor lead wires 504 and 506 include wire in a range of from 36 to 40 gauge wire.

The sensor coil 508 provides coil lead wire 518 at the coil distal end 514 and coil lead wire 516 at the coil proximal end 512. The two coil lead wires 516 and 518 electrically and mechanically couple the sensor coil 508 to the two sensor lead wires 504 and 506. The coil lead wire 516 is electrically and mechanically coupled to sensor lead wire 504, and the coil lead wire 518 is electrically and mechanically coupled to sensor lead wire 506. In some embodiments, at least one of the two coil lead wires 516 and 518 is coupled to the corresponding sensor lead wire 504 and 506 by soldering. In some embodiments, at least one of the two coil lead wires 516 and 518 is coupled to the corresponding sensor lead wire 504 and 506 by an adhesive. In some embodiments, at least one of the two coil lead wires 516 and 518 is coupled to the corresponding sensor lead wire 504 and 506 by laser wire attachment.

The sensor lead wire 506 is straight and runs parallel to the magnetic sensor coil 508 along the length of the magnetic sensor coil 508, from the coil distal end 514 to the coil proximal end 512, at lead wire portion 506a. The sensor coil 508 has a longitudinal axis Cx5 and the two sensor lead wires 504 and 506 extend proximal the sensor coil 508 in a twisted pair at twisted pair portion 506b. The twisted pair at the twisted pair portion 506b reduces sensor signal noise. In other embodiments, the sensor lead wires 504 and 506 do not include the twisted pair at 506b, instead, the sensor lead wires 504 and 506 are straight lead wires proximal the magnetic sensor coil 508 and shielded with a conductor, such as a braid of copper, which is advantageous for improving lead tensile strength and reducing sensor signal noise. This shielding can extend to the proximal end 512 or all the way to the distal end 514 of the magnetic sensor coil 508 and it can cover the connections between the coil lead wires 516 and 518 and the sensor lead wires 504 and 506. In some embodiments, the shield is electrically isolated from the sensor lead wires 504 and 506, the magnetic sensor coil 508, and/or the magnetic core 510.

The magnetic sensor assembly 500 includes an encapsulation material 520 that is wrapped around the magnetic sensor 502 and, in at least some embodiments, around portions of the two sensor lead wires 504 and 506. Optionally, an end cap 522 that is distal the coil distal end 514, is attached to the encapsulation material 520 to seal the encapsulation material 520 at the coil distal end 514. In some embodiments, the magnetic sensor 502 and the two sensor lead wires 504 and 506 are slid into a tube of encapsulation material 520. In some embodiments, the encapsulation material 520 is a shrink wrap material. In some embodiments, the sensor coil 508 and the sensor lead wire 506 are secured together, such as with an adhesive or plastic, at one or more locations along the sensor coil 508. In some embodiments, the end cap 522 includes at least one of epoxy, polyimide, and room temperature vulcanization silicone.

In assembling a catheter assembly such as ablation catheter assembly 24 of FIG. 1, the magnetic sensor assembly 500 is fit into the sensor lumen 50 and secured in place, such as with an adhesive. The coil distal end 514 is situated at the shaft distal end 46 and the two sensor lead wires 504 and 506 extend through the sensor lumen 50 to the shaft proximal end 44. The two sensor lead wires 504 and 506 are electrically coupled to the controller 34 at the shaft proximal end 44. The controller 34 receives the signals from the magnetic sensor 502 for determining the position of the catheter tip 22, where the magnetic sensor 502 provides the signals in response to externally applied magnetic fields.

Using a single coil magnetic sensor, such as magnetic sensor 502, the controller 34 can determine up to 5 DOF, including up/down, left/right, forward/backward, pitch, and yaw, based on the signals from the magnetic sensor 502. In other embodiments, where the magnetic sensor includes two or more magnetic sensor coils that are not parallel with each other along their longitudinal axes, the controller 34 can determine up to 6 DOF, including up/down, left/right, forward/backward, pitch, yaw, and rotation of the catheter tip 22 around its longitudinal axis Tx, based on the signals from the magnetic sensor.

Coupling the sensor lead wire 506 to the coil lead wire 518 at the coil distal end 514 may provide significant strain relief for the connections. By coupling the sensor lead wire 506 to the coil lead wire 518 at the coil distal end 514, instead of to the coil proximal end 512, the connection between the magnetic sensor 502 and the sensor lead wire 506 may be made more robust, such that it is not easily pulled apart or broken by stresses on the magnetic sensor 502 and the sensor lead wire 506. The encapsulation material 520 may increase the durability of the connections between the magnetic sensor 502 and the two sensor lead wires 504 and 506. These features reduce risk to the patient and improve product quality and profitability.

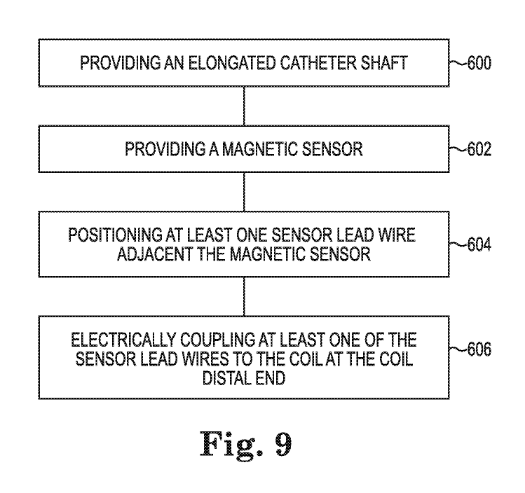

FIG. 9 is a flowchart diagram illustrating a method of assembling a catheter assembly, such as the ablation catheter assembly 20, according to embodiments described in the disclosure.

At 600, the method includes the step of providing an elongated catheter shaft having a shaft proximal end and a shaft distal end. The elongated catheter shaft includes lumens for wires, fluid, and a magnetic sensor assembly, such as one of the magnetic sensor assemblies 24, 100, 200, 300, 400, and 500 described in this disclosure. A catheter tip is connected to the shaft distal end.

A magnetic sensor assembly includes a magnetic sensor, a pair of sensor lead wires and, optionally, an encapsulation material. The magnetic sensor assembly is provided for insertion into the ablation catheter assembly. At 602, the method includes the step of providing a magnetic sensor. The magnetic sensor includes a sensor coil having a coil proximal end and a coil distal end that is situated toward or at the shaft distal end. The magnetic sensor also includes a magnetic core, where the sensor coil includes wire wrapped around the magnetic core in thousands of turns of wire.

At 604, the method includes the step of positioning at least one sensor lead wire adjacent the magnetic sensor, from the coil proximal end to the coil distal end. In some embodiments, positioning the at least one sensor lead wire includes positioning a straight portion of one or more of the sensor lead wires adjacent the magnetic sensor and a twisted portion of the sensor lead wires proximal the magnetic sensor. In some embodiments, positioning at least one sensor lead wire includes positioning each wire of the sensor lead wires adjacent another wire of the sensor lead wires and adjacent the magnetic sensor. In some embodiments, positioning at least one sensor lead wire includes positioning each wire of two sensor lead wires on opposing sides of the magnetic sensor and adjacent the magnetic sensor. In some embodiments, positioning at least one sensor lead wire includes wrapping one or more of the sensor lead wires around the sensor coil.

At 606, the method includes the step of electrically coupling at least one of the sensor lead wires to the coil at a coil lead wire at the coil distal end. In some embodiments, the sensor lead wires are electrically coupled to the coil at coil lead wires at the coil distal end by one of soldering, adhesives, and laser wire attachment. Also, in some embodiments, the method includes the step of encapsulating the magnetic sensor and the sensor lead wires in encapsulation material.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

I claim:

1. A catheter assembly, comprising:
an elongated catheter shaft having a shaft proximal end and a shaft distal end;
a magnetic sensor including a coil having a coil proximal end and a coil distal end that is situated toward or at the shaft distal end; and
one or more wires extending through the elongated catheter shaft and situated adjacent the magnetic sensor, the one or more wires electrically coupled to the coil at the coil distal end.

2. The catheter assembly of claim 1, comprising a controller electrically coupled to the one or more wires toward or at the shaft proximal end, the controller configured to determine the location of the shaft distal end in a patient based on signals from the magnetic sensor.

3. The catheter assembly of claim 1, wherein the coil has a longitudinal axis from the coil proximal end to the coil distal end and each of the one or more wires has a straight portion that is parallel to the longitudinal axis of the coil from the coil proximal end to the coil distal end.

4. The catheter assembly of claim 1, wherein the one or more wires are adjacent each other and the magnetic sensor.

5. The catheter assembly of claim 1, wherein the one or more wires are adjacent the magnetic sensor and on opposing sides of the magnetic sensor.

6. The catheter assembly of claim 1, wherein the one or more wires are twisted around or spiral around the coil from the coil proximal end to the coil distal end.

7. The catheter assembly of claim 1, wherein the one or more wires are twisted around each other proximal the magnetic sensor.

8. The catheter assembly of claim 1, wherein the one or more pair of wires are shielded straight wires that include a shield proximal the magnetic sensor.

9. The catheter assembly of claim 1, comprising a core having a high magnetic permeability, wherein the coil is wrapped around the core.

10. The catheter assembly of claim 1, comprising at least one of:
encapsulation material that encapsulates the one or more wires and the magnetic sensor in the encapsulation material; and
an end cap that is distal the coil distal end and attached to the encapsulation material.

11. A catheter assembly, comprising:
an elongated catheter shaft having a shaft proximal end and a shaft distal end;
a magnetic sensor including a coil having a longitudinal axis from a coil proximal end to a coil distal end that is situated at the shaft distal end; and
a pair of wires extending through the elongated catheter shaft, from the shaft proximal end to the shaft distal end, wherein each of the pair of wires has a straight portion that is adjacent the coil and parallel to the longitudinal axis of the coil along the length of the magnetic sensor, the pair of wires electrically coupled to the coil at the coil distal end.

12. The catheter assembly of claim 11, wherein the pair of wires are adjacent each other and one side of the magnetic sensor.

13. The catheter assembly of claim 11, wherein the pair of wires are adjacent the magnetic sensor and on opposing sides of the magnetic sensor.

14. The catheter assembly of claim 11, wherein the pair of wires are twisted around each other proximal the magnetic sensor.

15. The catheter assembly of claim 11, comprising encapsulation material that encapsulates the pair of wires and the magnetic sensor in the encapsulation material.

16. A method of assembling a catheter assembly comprising:
providing an elongated catheter shaft having a shaft proximal end and a shaft distal end;
providing a magnetic sensor including a coil having a coil proximal end and a coil distal end that is situated toward the shaft distal end;
positioning at least one of a pair of wires adjacent the magnetic sensor from the coil proximal end to the coil distal end; and
electrically coupling the at least one of the pair of wires to the coil at the coil distal end.

17. The method of claim 16, wherein positioning at least one of a pair of wires comprises twisting the at least one of the pair of wires around the magnetic sensor.

18. The method of claim 16, wherein positioning at least one of a pair of wires comprises positioning a straight portion of each of the wires adjacent the magnetic sensor and a twisted portion of the pair of wires proximal the magnetic sensor.

19. The method of claim 16, wherein positioning at least one of a pair of wires comprises positioning each wire of the pair of wires adjacent the other wire and adjacent the magnetic sensor.

20. The method of claim 16, wherein positioning at least one of a pair of wires comprises positioning each wire of the pair of wires on opposing sides of the magnetic sensor and adjacent the magnetic sensor.
